(19) United States
(12) Patent Application Publication
(10) Pub. No.: US 2002/0115143 A1
(43) Pub. Date: Aug. 22, 2002

(54) Rhabdovirus-based vectors to express high levels of functional human antibodies

(76) Inventors: Bernhard Dietzschold, Newtown Square, PA (US); Matthias J. Schnell, Harleysville, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/924,112

(22) Filed: Aug. 7, 2001

Related U.S. Application Data
(60) Provisional application No. 60/227,644, filed on Aug. 24, 2000.

(51) Int. Cl. C12P 21/02; C07K 16/00; C12N 15/86; C12N 5/06

(52) U.S. Cl. 435/69.1; 435/320.1; 435/456; 435/326; 530/388.1

(57) ABSTRACT

The current expression systems for the production of a specific antibody are time consuming, inadequate and costly. The present invention describes a novel recombinant expression vector using non-segmented, negative-stranded RNA virus vectors to express functional antibody. A high yield of pure antibody is obtained from this expression system and is used to neutralize the effect of an antigen. For example, antibody is used in prophylactic therapeutics, as well as in the treatment of an existing diseased condition.

### FIELD OF THE INVENTION

The present invention relates to the field of virology, and more particularly to immunology, wherein a recombinant, non-segmented negative stranded RNA virus serves as a vector for the expression of functional human antibodies for use in the treatment of infectious diseases or cancer.

### BACKGROUND OF THE INVENTION

It has been well over a decade since Cohen and Boyer first reported the use of bacterial plasmids as molecular cloning vectors; this marked the beginning of a new era in molecular biology and, historically, laid the foundation for what has been termed the “biotechnology industry.” Cohen and Boyer's cloning vector, pSC101, relative to the cloning vectors in circulation today, seems almost quaint—insertion of foreign DNA fragments into pSC101 was limited to a single restriction enzyme cleavage site and to Escherichia coli as a host. During the past decade, the art has had access to hundreds of molecular cloning vectors having nearly as much applicability and diversity as the number of vectors. Irrespective of the variety of such vectors, the typical objective remains the same: increased availability of a protein of interest that ordinarily is produced naturally in minute quantities.

In accordance with a typical strategy involving recombinant DNA technology, a DNA sequence which encodes a desired protein material (“cDNA”) is identified and either isolated from a natural source or synthetically produced. This piece of genetic material is ligated into a section of a small circular molecule of double stranded DNA. This circular molecule is typically referred to as a “DNA expression vector”.

The combination of the vector and the genetic material is referred to as a “recombinant”. The recombinant is isolated and introduced into a host cell and when the cellular DNA of the host cell replicates, the recombinant expression vector will also replicate. Accordingly, as the host cells grow and divide, there is a corresponding increase in cells containing the recombinant, which leads to the production (“expression”) of the protein material of interest. By subjecting the host cells containing the recombinant to favorable growth conditions, significant amounts of the host, and hence the protein of interest, are produced.

The vector plays a crucial role in determining the conditions under which expression of the genetic material will or will not occur. However, most of the vector manipulations are geared toward a single goal—increasing expression of a desired gene product, ie protein of interest. Stated again, most vector manipulation is conducted so that an “improved” vector will allow for production of a gene product at significantly higher levels when compared to a “non-improved” vector. Thus, while certain of the features/aspects/characteristics of one vector may appear to be similar to the features/aspects/characteristics of another vector, it is often necessary to examine the result of the overall goal of the manipulation—improved production of a gene product of interest.

A characteristic desirable for vectors is increased efficiency, that is, the ability to increase the amount of protein of interest. Such increased efficiency has several desirable advantages, including, but not limited to, reducing manufacturing costs and increasing purity of the protein product. Accordingly, there is a long sought need to significantly improve the current state of the art by developing expression vectors with such efficiency characteristics. The present invention has filled this long sought need by developing chimeric rabies virus expression vectors that express functional human antibodies.

Human rabies is a worldwide public health problem. Nearly half a million people receive annually rabies post-exposure prophylaxis (Steele, Rev. Infect. Dis. 10 (Suppl. 4): 585, 1988) which includes the use of anti-rabies virus immunoglobulin together with the administration of rabies vaccine (Wilde et al.,Vaccine 7: 478, 1989). Equine anti-rabies immunoglobulin (ERIG) and human anti-rabies immunoglobulin (HRIG) which are currently used for rabies post-exposure prophylaxis are either associated with severe adverse effects or are, as in the case of HRIG, extremely expensive. There are also safety concerns for HRIG because it is prepared from pooled human sera and, therefore, could be potentially contaminated with human pathogens. The present invention provides for an alternative for the production of monoclonal antibodies by the insertion of the nucleotide sequences coding for heavy and light chains of these human monoclonal antibodies into suitable expression vectors and expressing the inserted protein-coding sequences in appropriate cells, preferably eukaryotic cells.

As a first step toward the production of safer reagents, several human monoclonal antibodies (h MAbs) to rabies virus have recently been made by fusion of Epstein-Barr virus (EBV)-transformed rabies virus- specific human B cells with mouse-human heterohybrid cells (Ueki, et al., J. Exp. Med. 171: 19, 1990; Champion, et al., J. Immunol. Methods 235: 81, 2000). Several of these human monoclonal antibodies neutralized a broad spectrum of rabies virus and were able to protect hamsters against a lethal rabies virus infection when administered after infection (Dietzschold, B., et al., J. Virol. 65: 3087, 1990) indicating their great utility for the rabies post-exposure treatment of humans. However, in order to be routinely used in rabies treatment, large quantities of these human monoclonal antibodies must be cost effectively produced. Because of low expression levels (˜1 mg/l) and instability, the use of mouse-human heterohybrid cells secreting these human monoclonal antibodies, is not feasible for mass production.

There is a great need for safer and more effective products, for example for the post-exposure prophylaxis of human rabies. The evidence of the present invention indicates that rabies virus neutralizing human monoclonal antibodies will replace the currently used human anti-rabies immunoglobulin (HRIG) or equine anti-rabies immunoglobulin (ERIG) as a safer and more effective treatment. The main advantages of these human monoclonal antibodies over HRIG or ERIG are high specific protective activity, invariability of biological activity, and lack of risk and adverse effects.

Hybridoma technology for production of human monoclonal antibodies has become relatively easy and several mouse-human heterohybrid cell lines that secrete rabies virus neutralizing human monoclonal antibodies have already been established (Ueki, et al., J. Exp. Med. 171: 19, 1990; Champion, et al., J. Immunol. Methods 235: 81, 2000). The problem regarding a cost effective production of human Monoclonal antibodies is overcome by taking advantage of recombinant DNA technology of the present invention.

The expression vector disclosed herein allows for a high yield production of functional antibody. Although antibodies require extensive post-translational processing to become bioactive, several mouse and human immunoglobulin (Ig) heavy (H) chain and light (L) chain genes have been cloned and recombined with a variety of vectors which were able to express functional antibodies in eukaryotic expression systems. The eukaryotic expression systems currently used include lymphoid and non-lymphoid mammalian cells (Ovens, R. J. and Young, R. J., J. Immunolo. Meth., 168, 149-165, 1994), insect cells (Liang, et al, Virol. 235, 252-260, 1997), and plants (Whitelam, et al, Biochem. Soc. Transactions, 22, 940-944, 1994). While some of these expression systems, in particular mouse myeloma cells transfected with plasmid vectors containing Ig H and Ig L chain genes, are able to produce high levels of antibody, the recombinant expression vector (SPBN, see FIG. 1) of the present invention offers several advantages. The modular genome organization of the SPBN vector readily allows genetic manipulations and insertion of Ig H and Ig L chain genes. In contrast, the current state of the art uses transfection and selection of stable antibody expressing cell lines, which is a time consuming process.

The genome of the SBPN vector is a negative sense single-stranded RNA, thus expression of foreign genes is very stable and recombination events do not occur. In comparison, many myeloma cells often undergo somatic hypermutation and, therefore, have to be constantly recloned to maintain expression of the antibody.

The viral expression vectors used to date are used only in a very few cell types. In contrast, the SPBN vector of the present invention is extremely versatile. Because it contains the Vesicular Stomatitis Virus (VSV) glycoprotein (G), this vector is polytropic and able to infect and replicate in almost every mammalian or avian cell.

Further, many DNA and RNA viruses that express antibody are cytopathic, thereby limiting the expression, and hence yield, of antibody. The SPBN vector is non-cytopathic and, therefore, allows infected cells to produce antibody over a long period of time.

Transfected myeloma cells, the current cell line used for expression of functional antibody, must replicate to high numbers in order to produce a large scale production of antibody. The SPBN expression vector of the present invention allows for a high number of tissue culture cells to be infected simultaneously, enabling production of large amounts of antibody within a short period of time. Therefore, the SPBN expression system is well suited for industrial antibody production.

Definitions

immunoglobulin means antibody

### SUMMARY OF THE INVENTION

The present invention relates to an expression vector wherein a recombinant non-segmented negative-stranded RNA virus expresses a cDNA encoding an immunoglobulin. In one embodiment the immunoglobulin is a heavy chain. In another embodiment the immunoglobulin is a light chain. In another embodiment the cDNA encodes an immunoglobulin heavy chain and an immunoglobulin light chain. The present invention further relates to a method for expressing a functional immunoglobulin. A mammalian cell is infected with an expression vector wherein a recombinant non-segmented negative-stranded RNA virus expresses immunoglobulin heavy and light chains. The supernatants from the tissue culture cells are harvested, the virus is inactivated, and the supernatants are tested for the presence of neutralizing antibody.

A further embodiment of the present invention is a method for expressing a functional immunoglobulin wherein a mammalian cell is double-infected with expression vectors. One expression vector is a recombinant non-segmented negative-stranded RNA virus vector expressing an immunoglobulin heavy chain and the other expression vector is a recombinant non-segmented negative-stranded RNA virus vector expressing an immunoglobulin light chain. The supernatants from the mammalian cell tissue cultures are harvested, the virus is inactivated, and the supernatants are tested for the presence of neutralizing antibody. It is a further object of the present invention to present a method of treating a condition in which an antigen is recognized. A therapeutically effective amount of a purified antibody is administered to a mammal. The antibody binds to the antigen, thereby preventing a diseased state from persisting. In another embodiment the condition in which an antigen is recognized is treated by administering a therapeutically effective amount of a purified viral vector wherein a recombinant non-segmented, negative-stranded RNA virus vector expresses an antibody. The antibody is expressed in vivo and binds to the antigen, preventing a diseased state from persisting. The present invention further relates to a method of prophylactically preventing a condition in which an antigen is recognized. A therapeutically effective amount of a purified antibody is administered; the purified antibody binds to the antigen and prevents a diseased state from occurring. Another embodiment relates to administration of a therapeutically effective amount of a purified viral vector, wherein a recombinant non-segmented, negative-stranded RNA virus vector expresses an antibody. The antibody binds to the antigen and prevents a diseased state from occurring.

### DESCRIPTION OF THE DRAWINGS

FIG. 1. Schematic representation of the construction of the SPBN vector, expressing human IgG antibody genes. The glycoprotein (G) gene of rabies virus (RV) was replaced with a chimeric glycoprotein (G) which contains the ecto- and transmembrane domain of VSV glycoprotein fused to the cytoplasmic domain of RV glycoprotein. To obtain SPBN vectors expressing human IgG antibody the pseudo gene of RV (ψ) was replaced by the genes encoding the light (IgG 1), heavy (IgG h), or both light and heavy Ig chain resulting in the vectors SPBN−H, SPBN−L, and SPBN−H+L.

FIG. 2. A. Protein A Sepharose chromatography of human anti-rabies antibody JA-3.3A5 expressed in BSR cells by SPBN−H+L. The dashed line shows the protein concentration and the solid line the virus neutralizing titers in international units. (I.U.). B. A polyacrylamide gel electrophoresis of 1/100 volume of the eluted fraction of human anti-rabies antibody JA-3.3A5 (FIG. 2A). The gel was stained with Coomassie Brillant Blue to visualize the protein bands.

### DESCRIPTION OF THE INVENTION

Growing evidence suggests that certain human antibodies play an essential role in controlling infection, such as rabies virus (RV) or human immunodeficiency virus (HIV), (Baba, et al, Nature Medicine, 6:200-206, 2000), Habel, K., Bulletin of the World Health Organization, 38:383-7,1997), Serokowa, et al, przeglad Epidemiologiczny, 23:481-8,1969) Sziegoleit, A. and H. J. Gerth, Medizinische Mikrobiologie und Parasitologie, 218:24-31, (1971). In addition, it has been shown that antibodies directed against cancer cells play an important role in the therapy of a cancer patient (Gramatzki, M. and T. Valerius, Internist, 38:1055-62, 1997) Kishore, et al, Journal of the Association of Physicians of India, 26:479-84, 1978), (Ward, et al, lessons from the clinic Cancer Treatment Reviews, 23:305-19, 1997). The present invention is a new method to express a human antibody directed against RV glycoprotein (G) [JA-3.3A5 (3)] by a chimeric rabies virus. This vector allows for the expression of functional human immunoglobulin (IgG) heavy and light chains, as shown by rabies virus neutralization assays (see, infra). The data indicate that functional human antibodies are expressed by chimeric rabies viruses. This method is readily applied to the production of other antibodies, including, but not limited to, those directed against HIV or other Rhabdoviruses for example.

Antibody molecules bind to ligands with high affinity and specificity, the ability to discriminate between the epitope to which it is directed and any other epitope, makes them ideal immunotherapeutic agents. Immunotherapy includes, but is not limited to, treatment (prophylactic or post-exposure) for infectious agents, allergens, cancer, or any other condition in which an antigen is recognized.

The present invention is directed to recombinant, non-segmented, negative-stranded RNA virus vectors expressing a human antibody, the antibody is directed against any known antigen. In one embodiment of the invention the antibody is purified away from infected cells. In another embodiment of the present invention the viral vector expressing a human antibody is used. Both the purified antibody, as well as the viral vector expressing antibody, are used for prophylactic or post-exposure treatment of infectious diseases, for treatment of cancers or for any condition in which an antigen is recognized.

cDNA Cloning of Human IgG Heavy and Light Chains from JA-3.3A5 Hybridoma Cell

Total RNA was isolated from JA-3.3A5 hybridoma cell by using RNAzol B (Biotecx Laboratories, Houston). Reverse transcriptase reactions were performed at 42° C. for lhr with avian myeloblastosis virus reverse transcriptase (Promega) and oligo(dT) primer. A portion of the RT products were subjected to polymerase chain reaction (PCR) amplification using heavy chain specific primers: IgG-HF1 primer (5′-ACCATGGAGTTTGGGCTGAG-3′ (SEQ. ID. NO: 1); start codon of heavy chain underlined, (gene bank accession # Y14737), and IgG-HR1 primer (5′-ACTCATTTACCCGGGGACAG-3′ (SEQ. ID. NO: 2); stop codon of heavy chain underlined, (gene bank accession # Y14737) or light chain specific primers: IgG-LF5 primer (5′-AGCATGGAAGCCCCAGCTCA-3′ (SEQ. ID. NO: 3); start codon of light chain underlined, (gene bank accession # M63438), and IgG-LR2 primer (5′-CTCTAACACTCTCCCCTGTTG-3′ (SEQ. ID. NO: 4); stop codon of light chain underlined, (gene bank accession # M63438). Amplification was carried out for 35 cycles of denaturation at 94° C. for 60 sec, annealing at 50° C. for 60sec, and elongation at 72° C. for 90 sec with Taq DNA polymerase (Promega). The PCR products (1.4 kb for heavy chain, 0.7 kb for light chain) were purified and sequenced by using the AmpliTaq cycle sequencing kit (Perkin-Elmer) with the specific primers. The PCR products were cloned into TA cloning vector, pCR2.1 (Invitrogen). The cloned heavy chain and light chain sequence was confirmed by DNA sequencing.

Construction of Recombinant Rabies Virus Clones Containing Human IgG Heavy and Light Chains.

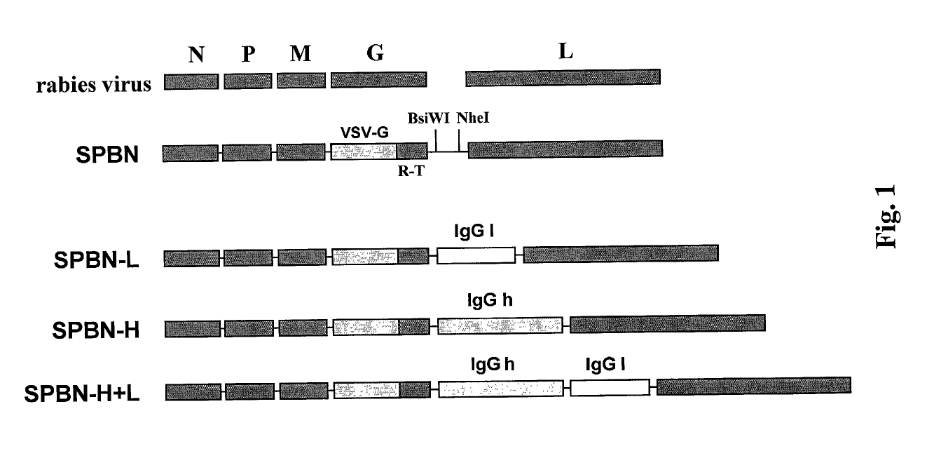

The human antibody that is expressed is directed against rabies virus (RV) glycoprotein, therefore a modified version of the previously described rabies virus expression vector (Schnell, et al, Proc. Natl. Acad. Instit. Sci. USA 97: 3544-3549, 2000) which contains a chimeric Vesicular Stomatitis virus (VSV)/rabies virus glycoprotein (G) is used in the present invention. This chimeric glycoprotein contains the ecto- and transmembrane domain of VSV glycoprotein fused to the cytoplasmic domain of RV glycoprotein (SPBN, FIG. 1).

IgG heavy chain cDNA was amplified by PCR using Vent polymerase (New England Biolabs) and primers IgG H BsiWI (5′-AACGTACGACCATGGAGTTTGGGCTGAGCT-3′ (SEQ. ID. NO: 5); BsiWI site in bold face, the start codon underlined) and IgG H Nhe (5′-AAGCTAGCTCATTTACCCGGGGACAGGGAG-3′ (SEQ. ID. NO: 6); NheI site in bold face, the stop codon underlined). For IgG light chain cDNA, IgG L BsiWI (5′-AACGTACGAGCATGGAAGCCCCAGCTCAGC-3′ (SEQ. ID. NO: 7); BsiWI site in bold face, the start codon underlined) and IgG L Xba (5′-GGTCTAGACTAACACTCTCCCCTGTTGAAG-3′ (SEQ. ID. NO: 8); NheI site in bold face, the stop codon underlined) were used. PCR products were digested with BsiWI and NheI (for heavy chain cDAN), or BsiWI and XbaI (light chain cDNA), and ligated to pSPBN, which had been digested with BsiWI and NheI, or BsiWI and XbaI, respectively. The resulting plasmids were designated pSPBN-H (heavy) and pSPBN-L (light).

A recombinant RV expressing both the heavy and light chains from one viral genome was constructed. The coding region of the light chain, INT5(+), was amplified by PCR using the primers ITN5(+) (5′-CTGTCTCCGGGTAAATGAGTCATGAAAAAAACTAACACCCCTAGCATGGA AGCCCCAGCTCA-3′ (SEQ. ID. NO: 9) [stop codon of the heavy chain and start codon of the light chain italicized, rabies virus transcription stop/start signal underlined] and IgG-LR2 (SEQ. ID. NO: 4). The coding region of the heavy chain was amplified by PCR using the primers INT3(−) TGAGCTGGGGCTTCCATGCTAGGGGTGTTAGTTTTTTTCATGACTCATTTA CCCGGAGACAG-3′ (SEQ. ID. NO: 10) and IgG-HF1 (SEQ. ID. NO: 1). Both PCR products were annealed, and amplified by PCR using Vent polymerase and primers IgG H Bsi (SEQ. ID. NO: 5) and IgG L Xba (SEQ. ID. NO: 8) primers. The 2.1 kb PCR product was digested with BsiWI and XbaI, and ligated to pSPBN. The resulting plasmids were designated as pSPBN−H+L.

Recovery of Recombinant Rabies Virus

Recombinant viruses free of vaccina virus were rescued as described (Finke, S. and K. K. Conzelmann, Journal of Virology, 73:3818-25, 1999; Schnell, et al, Proc. Natl. Acad. Instit. Sci. USA 97: 3544-3549, 2000). Briefly, BSR-T7 cells (Buchholz, Journal of Virology, 73:251-9, 1999) were grown overnight to 80% confluency in 6-well plates in DMEM supplemented with 10% FBS. One hour before transfection, cells were washed twice with serum-free DMEM. Cells were transfected with 5.0 μg of full-length plasmid, 5.0 μg of pTIT-N, 2.5 μg of pTIT-P, 2.5 μg of pTIT-L, and 2.0 μg of pTIT-G (Finke, S. and K. K. Conzelmann, Journal of Virology, 73:3818-25, 1999), using a CaPO₄ transfection kit (Stratagene, La Jolla, Calif.). After 2-3 h, cells were washed twice and maintained in DMEM supplemented with 10% FBS for 3 days. The culture medium was transferred onto BSR cells and incubated for 3 days at 34° C. The BSR cells were examined for presence of rescued virus by immunofluorescence assay with FITC-labeled rabies virus N protein-specific antibody.

These passages were repeated to replenished with serum-free medium supplemented with 0.2% bovine serum albumin, and incubated at 37° C.

The supernatant of positive cell cultures was infected into BSR cells, and 3-4 days later, the infected culture was passaged with 1:6 dilution. In each passage, the BSR cells were examined for presence of rescued virus by immunofluorescence. These passages were repeated to get a high yield of the virus. Rescued viruses generated from full-length plasmids; pSPBN-Heavy, pSPBN-Light, pSPBN−H+L were SPBN-Heavy, SPBN-Light, SPBN−H+L, respectively. Sequences of recombinant viruses were confirmed by sequencing of the RT-PCR fragments.

Cells and Viruses

Neuroblastoma NA cells of A/J mouse origin and murine myeloma cells (Sp2/o), were grown at 37° C. in RPMI 1640 medium supplemented with 10% heat-inactivated fetal bovine serum (FBS). Chinese hamster ovarian cells (CHO), BSR cells, a cloned cell line derived from BHK-21 cells, and BSR-T7 cells, a cell line derived from BSR cells which constitutively express T7 RNA polymerase (1), were grown at 37° C. in Dulbecco's modified Eagle's medium (DMEM) supplemented with 10% heat-inactivated FBS. Mouse-human heterohybrid cell producing human monoclonal antibody (h Mab) JA-3.3A3 were established as previously described (Champion, H J. M., et al., J. Immunol. Methods 235:81, 2000).

CVS-N2c and CVS-B2c are subclones of the mouse-adapted CVS-24 rabies virus (Morimoto et al., Proc. Nati. Acad. Sci. USA 95: 3152, 1998). SHBRV-18 and DRV-4 are street rabies virus strains associated with silver-haired bats or dogs, respectively (Dietzschold et al., J. Hum. Virol. 3:50, 2000). SN-10 is a non-pathogenic virus strain derived from SAD B 19 (Schnell et al., Proc. Natl. Acad. Sci. USA 97:3544, 1994).

Virus Infection and Virus Titration

Cells were infected with the different recombinant viruses at a m.o.i. of 1.0 and incubated for 1 hr at 37° C. Then the cells were washed twice with RPMI 1640 or DMEM, replenished with serum-free medium supplementd with 0,2% bovine serum albumin, and incubated at 37° C.

To determine the virus yield, monolayers of NA cells in 96-well plates were infected with serial 10-fold dilutions of virus suspension and incubated at 34° C. as described (Wiktor, et al., Biochem., Soc. Transactions 22: 940, 1994). At 48 hrs postinfection, cells were fixed in 80% acetone and stained with fluorescein isothiocyanate (FITC)-labeled rabies virus N protein-specific antibody (Centocor Inc. Malvern, Pa.). Foci were counted using a fluorescence microscope. All titrations were carried out in triplicate.

Virus Neutralization Test

Supernatant samples from infected cells were exposed to short wave UV light for 20 minutes to inactivate the virus and then tested for presence of virus neutralizing antibody using the rapid fluorescent inhibition test (RFFIT) as previously described (Wiktor, et al., Dev. Biol. Stand. 57: 199, 1984). The virus-neutralizing antibody (VNA) titer was normalized to international units (I.U.) using the World Health Organization (WHO) anti-rabies virus antibody standard.

Purification of Antibody by Affinity Chromatography

Recombinant human monoclonal antbody (r h Mab) was purified using a protein A column (rProtein A Sepharose™ Fast Flow, Amersham Pharmacia Biotech). Briefly, supernatants were clarified by filtration through a 0.45 μm membrane and the pH adjusted to 8.0 with 1N NaOH. Supernatant was run through the column at a linear flow rate of approximately 100 ml/hr. To destroy infectious virus and to remove viral and cellular contaminants , the column was washed with PBS containing 1% Triton X 100 followed by PBS alone, and antibody was eluted from the column using a 0.1M citric acid, pH 3.0. Two ml fractions were collected, each fraction dialyzed against PBS, and protein concentrations were determined using the protein detection assay (Bio-Rad Laboratories, Hercules Calif.) according to the manufacturer's instructions.

Polyacrylamide Gel Electrophoresis

A twenty μl aliquot from each fraction eluted from the protein sepharose column was mixed with an equal volume of loading buffer (100 mM Tris-HCl, pH 6.8, 200 mM dithiothreitol, 4% SDS, 0.2% bromophenol blue, 20% glycerol) and subjected to electrophoresis on an SDS-10% SDS polyacrylamide gel. To visualize protein bands, the gel was stained with Coomassie Brillant Blue.

Results

Antibody production in tissue cultures infected with SPBN−L SPBN−H, and SPBN−H+L

Immunofluorescence analysis using FITC-conjugated antibodies specific for human kappa chains or human IgG 1 revealed that the genes encoding Ig H chain and Ig L chain are expressed in BSR cells infected with SPBN−H and SPBN−L, respectively. BSR cells ifected with SPBN−H+L expressed both, Ig H chain and Ig L chain.

To determine whether functional antibodies are expressed by the chimeric rhabdovirus vector SPBN-SN, monolayers of mouse neuroblastoma (NA) cells, BSR cells, CHO cells and Sp2/0 cells were infected at a multiplicity of infection (m.o.i). of 1.0 with SPBN−H+L or double-infected with SPBN−H and SPBN −L, each at a multiplicity of infection of 1.0. Six days after infection, the tissue culture supernatants were harvested and exposed to shortwave UV light for 20 min to inactivate the virus and then tested for presence of virus neutralizing antibody.

Virus neutralization was performed using the fluorescent focus inhibition test and employing CVS, a highly pathogenic rabies virus, as challenge virus and neuroblastoma cells as indicator cells. The titer was normalized to international units (I.U.) using the World Health Organization (WHO) anti-rabies virus antibody standard. Table 1 shows that while no virus-neutralizing activity was detected in the supernatant of NA, BSR, CHO, or SP2/0 (murine myeloma cells) cells infected with the SPBN vector, the supernatant of NA or BSR cells infected with either SPBN−H+L or double infected with SPBN−L and SPBN−H contained rabies virus-neutralizing activity. The highest virus neutralizing titer was detected in the supernatant of BSR cells infected with SPBN−H+L. Comparison of virus neutralizing antibody (VNA) titers with virus titers indicate that the level of antibody production in SPBN-H+L-infected cells correlates with the virus titer produced by these cells.

TABLE 1
Expression of rabies virus neutralizing monoclonal antibody
JA-3.A3 by the rhabdovirus-based SPBN vector

| | | | Vector | | |
|---|---|---|---|---|---|
| | Vector | | SPBN-H | | |
| | SPBN-H + L | | SPBN-L | | |
| | Virus titer | VNA titer | Virus titer | VNA titer | Vector |
| | FFU/ml | IU/ml | FFU/ml | IU/ml | SPBN |
| NA | 2 × 10⁶ | 0.36 | ND | 0.27 | 0 |
| BSR | 2 × 10⁷ | 2.84 | ND | 0.36 | 0 |
| CHO | 1.5 × 10⁴ | 0.15 | ND | ND | 0 |
| Sp 2/0 | 5.5 × 10⁵ | 0.15 | ND | ND | 0 |

ND: not done

Purification and Electrophoretic Analysis of the Antibody Expressed by SPBN−H+L

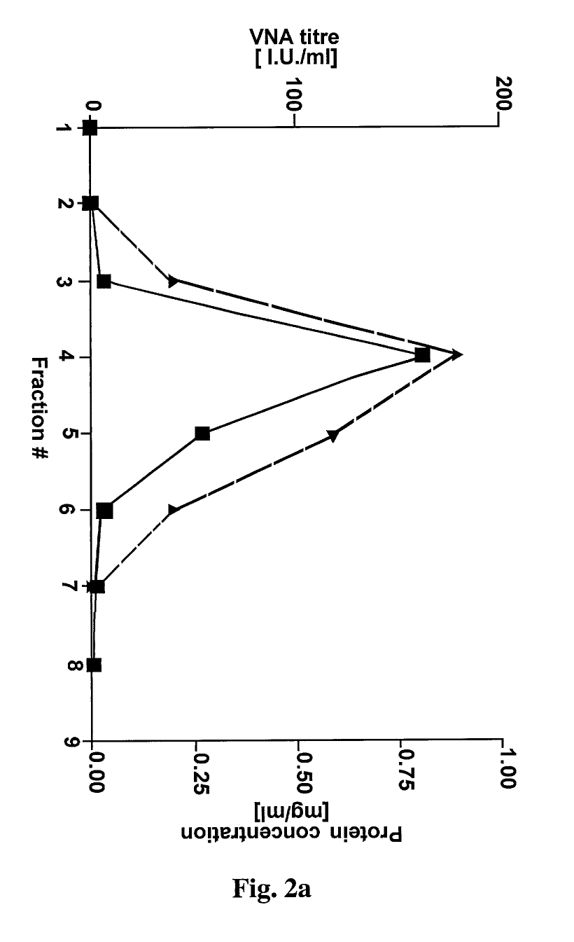

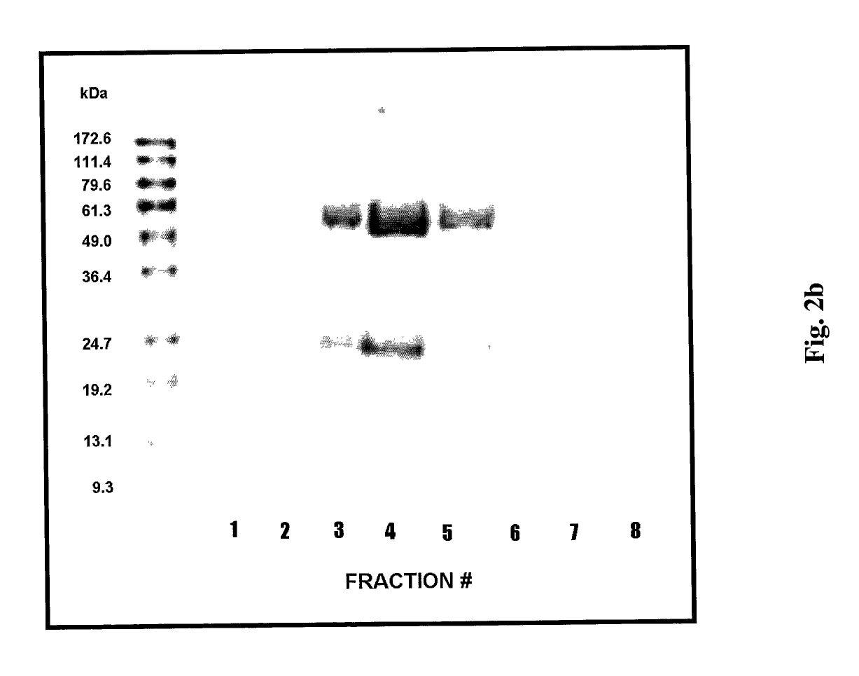

To determine whether intact antibody molecules containing both light and heavy chain are secreted into the tissue culture supernatant, 350 ml supernatant harvested from 5×10⁸ SPBN−H+L-infected BSR cells 6 days after infection were subjected to chromatography on a Protein A Sepharose column. After adsorption, the column was washed with PBS and the adsorbed antibody eluted with 0.25 M citric acid, pH 3.0. Two ml fraction were collected and an aliquot (20 μl) of each fraction subjected to SDS-polyacrylamide electrophoresis. Protein bands were visualized by staining with Coomasie blue. VNA testing and polyacrylamide gel electrophoresis (FIG. 2) demonstrates that the antibody, which is eluted in a sharp peak, consists of both light and heavy chain antibody. The amount of neutralizing antibody purified from the 350 ml tissue culture supernatant was 3.3 mg or 594 IU, indicating that SPBN−H+L expresses high levels of structurally and functionally intact antibody. Replenishing of the infected cells with serum-free medium followed by incubation for another 6 days resulted in a similar amount of antibody indicating that at least 19 mg of antibody is produced by 5×10⁸ cells, corresponding to 38 pg/cell /12 days.

Specificity of the Antibody Expressed by SPBN−H+L

To determine whether the recombinant antibody rJA-3.3A5 expressed by SPBN−H+L exhibits the same specificity as that of the parental mouse-human heterohybrid antibody JA-3.3A5 both antibody preparations were adjusted to the same protein concentration (0.5 mg/ml) and compared for their ability to neutralize different rabies virus strains (Table 2). While the VNA titers against SN-10 and SHBRV-18 were identical, the VNA titers of rJA-3.3A5 against CVS-N2c and DRV-4 were nine times higher as compared to the titers obtained with JA-3.3A5. On the other hand, VNA titers of rJA-3.3A5 against CVS-B2c were somewhat lower than those obtained with JA-3.3A5.

TABLE 2
Comparison of the virus-neutralizing capacity of recombinant antibody
rJA-3.3A5 and parental mouse-human heterohybrid antibody JA-3.3A5

| | VNA titer(IU) | | | | |
|---|---|---|---|---|---|
| | Virus strain | | | | |
| Antibody* | CVS-B2c | CVS-N2c | SN-10 | DRV-4 | SHBRV-18 |
| JA3.3A5 | 1.3 | 12.0 | 4.0 | 18.0 | 4.0 |
| rJA3.3A5 | 0.4 | 108.0 | 4.0 | 162.0 | 4.0 |

*Antibodies were purified by Protein A Sepharose chromatography and adjusted to a protein concentration of 0.5 mg/ml.

Therapeutic and Prophylactic Methods and Compositions

The invention provides methods of treatment and prophylaxis by administration to a subject of an effective amount of a purified antibody or the viral vector expressing the antibody. The subject is preferably an animal, including but not limited to animals such as cows, pigs, chickens, etc., and is preferably a mammal, and most preferably human.

The purified antibody is administered by intravenous injection. The viral vector expressing the antibody is administered so that it becomes intracellular, (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (Joliot et al, Proc. Natl. Acad. Sci. U.S.A. 88:1864-1868, 1999).

The amount of the purified antibody, or viral vector expressing the antibody, of the invention which is effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will depend on the seriousness of the disease or disorder, and is decided according to the judgment of the practitioner and each patient's circumstances. Effective doses may be extrapolated from dose-response curves derived from in vitro or animal model test systems.

What is claimed is:

1. An expression vector, comprising a recombinant non-segmented negative-stranded RNA virus expressing a cDNA encoding an immunoglobulin heavy chain.

2. An expression vector, comprising a recombinant non-segmented negative-stranded RNA virus expressing a cDNA which encodes an immunoglobulin light chain.

3. An expression vector, comprising a recombinant non-segmented negative-stranded RNA virus expressing a cDNA which encodes an immunoglobulin heavy chain and an immunoglobulin light chain.

4. A method for expressing a functional immunoglobulin, comprising
a) infecting a mammalian cell with an expression vector, said expression vector comprising a recombinant non-segmented negative-stranded RNA virus vector expressing immunoglobulin heavy and light chains;
b) harvesting tissue culture supernatants;
c) inactivating virus;
d) testing for the presence of neutralizing antibody.

5. A method for expressing a functional immunoglobulin, comprising
a) double-infecting a mammalian cell with expression vectors, comprising a recombinant non-segmented negative-stranded RNA virus vector expressing an immunoglobulin heavy chain and a recombinant non-segmented negative-stranded RNA virus vector expressing an immunoglobulin light chain;
b) harvesting tissue culture supernatants;
c) inactivating virus; and
d) testing for the presence of neutralizing antibody.

6. A method of treating a condition in which an antigen is recognized, comprising:
a) administering a therapeutically effective amount of a purified antibody;
b) binding of said purified antibody to said antigen; and
c) preventing a diseased state from persisting.

7. A method of treating a condition in which an antigen is recognized, comprising:
a) administering a therapeutically effective amount of a purified viral vector, said vector comprising a recombinant non-segmented, negative-stranded RNA virus vector expressing an antibody;
b) binding of said antibody to said antigen; and
c) preventing a diseased state from persisting.

8. A method of prophylactically preventing a condition in which an antigen is recognized, comprising:
a) administering a therapeutically effective amount of a purified antibody;
b) binding of said purified antibody to said antigen; and
c) preventing a diseased state from occurring.

9. A method of prophylactically preventing a condition in which an antigen is recognized, comprising:
a) administering a therapeutically effective amount of a purified viral vector, said vector comprising a recombinant non-segmented, negative-stranded RNA virus vector expressing an antibody;
b) binding of said antibody to said antigen; and
c) preventing a diseased state from occurring.


SEQUENCE LISTING (10 sequences)

SEQ ID NO: 1 - DNA, 20 bp, Artificial Sequence
  PCR primers
accatggagtttgggctgag

SEQ ID NO: 2 - DNA, 20 bp, Artificial Sequence
  PCR primers
actcatttacccggggacag

SEQ ID NO: 3 - DNA, 20 bp, Artificial Sequence
  PCR primers
agcatggaagccccagctca

SEQ ID NO: 4 - DNA, 21 bp, Artificial Sequence
  PCR primers
ctctaacactctcccctgttg

SEQ ID NO: 5 - DNA, 30 bp, Artificial Sequence
  PCR primers
aacgtacgaccatggagtttgggctgagct

SEQ ID NO: 6 - DNA, 30 bp, Artificial Sequence
  PCR primers
aagctagctcatttacccggggacagggag

SEQ ID NO: 7 - DNA, 30 bp, Artificial Sequence
  PCR primers
aacgtacgagcatggaagccccagctcagc

SEQ ID NO: 8 - DNA, 30 bp, Artificial Sequence
  PCR primers
ggtctagactaacactctcccctgttgaag

SEQ ID NO: 9 - DNA, 62 bp, Artificial Sequence
  PCR primers
ctgtctccgggtaaatgagtcatgaaaaaaactaacacccctagcatggaagccccagctca

SEQ ID NO: 10 - DNA, 62 bp, Artificial Sequence
  PCR primers
tgagctggggcttccatgctaggggtgttagtttttttcatgactcatttacccggagacag